(19) United States
(12) Patent Application Publication
(10) Pub. No.: US 2022/0134127 A1
(43) Pub. Date: May 5, 2022

(54) THERAPEUTIC WEARABLE

(71) Applicant: Biothread LLC, Wayne, PA (US)

(72) Inventors: Daniel Bish, New York, NY (US); Lawrence A. Blaustein, Chagrin Falls, OH (US); Daniel Shuter, New York, NY (US); Jay Tapper, Wayne, PA (US)

(21) Appl. No.: 17/084,989

(22) Filed: Oct. 30, 2020

(51) Int. Cl. A61N 5/06

(52) U.S. Cl. CPC A61N 5/0613; A61N 2005/063; A61N 2005/0663; A61N 2005/0645; A61N 2005/0659; A61N 2005/0632

(57) ABSTRACT

A wearable for providing light therapy to a wearer includes a fabric panel, an optical fiber light source, a side-emitting optical fiber, and a light pipe. The side-emitting optical fiber is affixed to the fabric panel. The light pipe includes a proximal end, with respect to the optical fiber light source, optically coupled to the optical fiber light source and a distal end optically coupled to a light-receiving end of the side-emitting optical fiber.

### BACKGROUND

Clinical studies have demonstrated the ergogenic and prophylactic benefits of red and infrared light therapy. Red and infrared light have been found to increase blood flow to muscles and joints, which can create an anti-inflammatory response, in addition to providing increased pliability. Muscle and joint stiffness as well as soreness have been demonstrated to be significantly reduced while muscle contractile function is simultaneously improved by using red and infrared light.

Red and infrared light therapy is utilized by professional athletes in addition to being widely available in medical spas and physical therapy outlets. Likewise, at the very high end of the consumer market, light therapy awareness and usage has grown.

Building on this awareness, there has been many non-light-based wraps and sleeves that have invaded social media espousing anti-inflammatory and muscle recovery benefits. While affordable and accessible, these products seem to only provide temporary relief of symptoms with little to no recovery or preventative benefits.

Clothing made from light emitting fabrics is described in U.S. Pat. No. 4,234,907. This patent, however, describes such clothing as a fad item or as safety clothing to emit light outward when the wearer wishes to be seen by others. US 2007/0089800A1 discloses garment systems that include an integrated infrastructure for monitoring vital signs of an individual and for other monitoring purposes. Neither of the aforementioned patent documents discloses a therapeutic wearable for delivering light of a therapeutic wavelength toward the wearer.

### SUMMARY

In view of the foregoing, a wearable for providing light therapy to a wearer includes a fabric panel, an optical fiber light source, a side-emitting optical fiber, and a light pipe. The side-emitting optical fiber is affixed to the fabric panel. The light pipe includes a proximal end, with respect to the optical fiber light source, optically coupled to the optical fiber light source and a distal end optically coupled to a light-receiving end of the side-emitting optical fiber.

In the aforementioned wearable described above, the optical fiber light source can be one of a first optical fiber light source emanating light of a first wavelength and a second optical fiber light source emanating light of a second wavelength that is different than the first wavelength. The light pipe can be one of a first wavelength-dedicated light pipe, a second wavelength-dedicated light pipe, and a combined wavelength light pipe. The first wavelength-dedicated light pipe can be positioned between the first optical fiber light source and an upstream end of the combined wavelength light pipe. The second wavelength-dedicated light pipe can be positioned between the second optical fiber light source and the upstream end of the combined wavelength light pipe. The combined wavelength light pipe also includes a downstream end optically coupled with the side-emitting optical fiber.

For the wearable described in the preceding paragraph, the combined wavelength light pipe can have a core having a diameter that is at least two times a respective diameter of each respective core of both the first wavelength-dedicated light pipe and the second wavelength-dedicated light pipe. The wearable described in the preceding paragraph can further include at least one light pipe optical fiber optically coupled with the downstream end of the combined wavelength light pipe, where the light pipe optical fiber has lower light attenuation along its length as compared to the side-emitting optical fiber. The light pipe optical fiber can be affixed to the fabric panel. The light pipe optical fiber can have a smaller bending radius the combined wavelength light pipe. The combined wavelength light pipe can include a core having a diameter that is at least three times a diameter of a core of the light pipe optical fiber. The light pipe optical fiber and the side-emitting optical fiber can be about equal, e.g. ±10%, in outer diameter.

For the wearable described in the preceding paragraph, the at least one light pipe optical fiber can include a plurality of light pipe optical fibers, and respective light-entering ends of each light pipe optical fiber can be optically coupled with the combined wavelength light pipe at the downstream end. Also, respective light-exiting ends of each light pipe optical fiber can be optically coupled with respective light-receiving ends of each side-emitting optical fiber among a plurality of side-emitting optical fibers. Each end of at least one of the plurality of side-emitting optical fibers can be a light-receiving end that is butt coupled to the combined wavelength light pipe at the downstream end.

In any of the aforementioned wearables described above, the distal end of the light pipe can optically coupled to each end of the side-emitting optical fiber. Alternatively and for any of the aforementioned wearables described above, the side-emitting optical fiber can be one of a plurality of side-emitting optical fibers, and the distal end of the light pipe can be optically coupled to each end of each of the plurality of side-emitting optical fibers. In such an embodiment having a plurality of side-emitting optical fibers, the distal end of the light pipe can be butt coupled to each end of each of the plurality of side-emitting optical fibers.

In any of the aforementioned wearables described above, the side-emitting optical fiber can be one of a plurality of side-emitting optical fibers, and the light pipe can have a core having a diameter that is at least three times a respective diameter of each respective core of each of the plurality of side-emitting optical fibers.

In any of the aforementioned wearables described above, the light pipe can be one of an upstream light pipe and at least one downstream light pipe optically coupled to the upstream light pipe. The upstream light pipe can include an upstream end or a first end that coincides with the proximal end of the light pipe and a downstream end or a second end, and each downstream light pipe of the at least one downstream light pipe can include a respective light-entering end optically coupled to the downstream end or to the second end of the upstream light pipe. Where the side-emitting optical fiber is one of a plurality of side-emitting optical fibers, each downstream light pipe can include a light-exiting end optically coupled to a respective light-entering end of each side-emitting optical fiber of the at least one side-emitting optical fiber.

In any of the aforementioned wearables described above, the light pipe can be one of a plurality of light pipe optical fibers each affixed to the fabric panel. In addition or alternatively, the light pipe and the side-emitting optical fiber can be about equal, e.g. ±10%, in outer diameter.

### BRIEF DESCRIPTION OF THE DRAWINGS

FIG. 1 is a front view of an example of a therapeutic wearable in the form of a garment for providing light therapy to a wearer of the garment.

FIG. 2 is a plan view of panels laid flat and prior to being sewn together to make up the garment in FIG. 1.

FIG. 3 is a schematic view of a light pod and at least one side-emitting optical fiber and light pipe optical fiber for use in the garment depicted in FIGS. 1 and 2.

FIGS. 4 and 5 are perspective views of an integrally formed light pipe.

FIG. 6 is a cross-sectional view taken through line 6-6 in FIG. 3.

FIG. 7 is a cross-sectional view taken through line 7-7 in FIG. 3.

FIG. 8 is a close-up view of the circled portion in FIG. 2.

FIG. 9 is an alternative connection to that shown in FIG. 8.

FIG. 10 is an alternative to FIG. 2 showing a plan view of panels laid flat and prior to being sewn together to make up the garment in FIG. 1.

FIG. 11 is a schematic view of one side-emitting optical fibers and light pipe optical fiber for use in the garment depicted in FIG. 10.

FIG. 12 is a plan view of an alternative therapeutic wearable.

### DETAILED DESCRIPTION

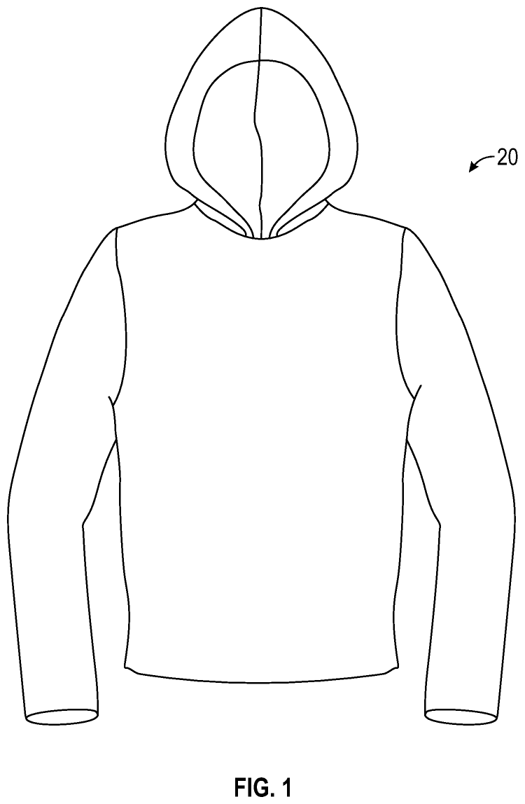

Red light having a wavelength between about 630 nm and about 700 nm has been found beneficial to increase blood flow and may provide ameliorative affects with regard to inflammation, as well as other health benefits. Infrared (“IR”) light having a wavelength between about 700 nm and about 1 mm has also been found to provide similar benefits. As such, light with these wavelengths can be referred to as light having a therapeutic wavelength. FIG. 1 depicts a light therapy wearable in the form of a garment 20 for providing light therapy to a wearer. The garment 20 is manufactured in a manner so as to project light having a therapeutic wavelength, which has been described above, toward a person wearing the garment 20. The garment 20 can be configured to project light toward targeted body areas, which can include particular muscles, muscle groups, joints, human extremities, and the wearer's skin as examples. The garment 20 shown in FIG. 1 is a hoodie, however, the garment 20 can be another type of garment, such as a shirt, shorts, pants, gloves, a hat, socks, and undergarment, a wrap, etc. The garment 20 is designed to be worn by a person in a similar manner as a conventional garment, so that the hoodie shown in FIG. 1 will be worn over the upper body of a person, for example.

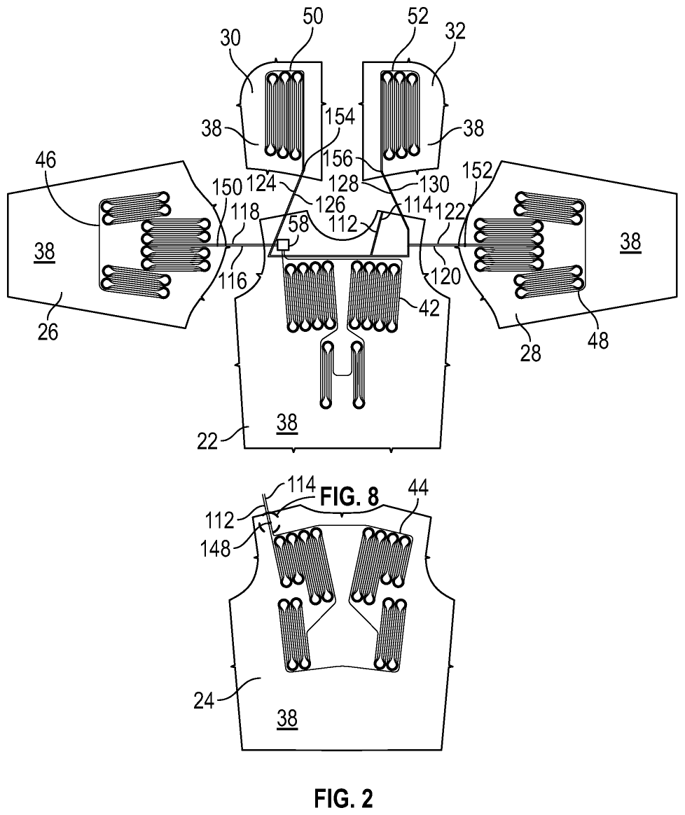

With reference to FIG. 2, the therapeutic garment 20 is made up of a plurality of panels 22 (front), 24 (back), 26 (right sleeve), 28 (left sleeve), 30 (right hood), 32 (left hood) that when sewn together to make up the therapeutic garment 20. FIG. 2 depicts the panels 22-32 prior to being sewn together to form the garment 20 shown in FIG. 1. FIG. 2 is a plan view showing an inner surface 38 of each panel, which is the surface facing the wearer when the garment 20 is worn. The panels 22-32 can be either cut and sew pattern pieces or fully fashioned knitted structures. Where the panels 22-32 are cut and sew pattern pieces, each panel 22-32 is shown in FIG. 2 after being cut from standard fabric. Where the panels 22-32 are fully knitted structures, each panel in FIG. 2 is shown after manufacturing each knitted structure. When the plurality of panels 22-32 are sewn together, openings are provided for the head and arms for the garment 20 shown in FIG. 1. Openings can be provided for legs of the wearer where the garment takes another configuration, such as shorts, pants or underwear.

In either the cut and sew pattern pieces or fully fashioned knitted structures, the yarn from which each panel 22-32 is made can provide a comfort component for the garment 20. Examples of such yarn can include cotton, polyester, cotton/polyester blends, microdenier polyester/cotton blends, and combinations thereof. It can be desirable to provide the garment 20 so that it is skin-tight or form-fitting to bring the therapeutic light source, which will be described in more detail below, very close to the wearer of the garment 20. Accordingly, the yarn can also include an elastic fiber such as lycra or spandex, and more than one type of yarn can form each panel.

Each of the panels 22-32 can be made from fabric having a four way stretch, i.e., each panel can have 100% or nearly 100% recovery along the grain and cross grain from 8% stretch. This can be desirable when the garment 20 is a shirt. Each of the panels 22-32 can be made from fabric having 100% or nearly 100% recovery along the grain and cross grain from 30% stretch, which can be useful when the garment is a sock or wrap, for example. When the panels are cut and sew pattern pieces, each of the panels 22-32 can be either woven or knitted. When the panels are fully fashioned knitted structures, each of the panels 22-32 are a knitted structure typically made with little or no extra fabric.

FIG. 2 depicts side-emitting optical fibers 42, 44, 46, 48, 50, 52 affixed to the inner surface 38 of each panel 22, 24, 26, 28, 30, 32 respectively. Each side-emitting optical fiber 42-52 is optically connectable with at least one optical light source, which will be described in more detail below, and is configured to project light having a therapeutic wavelength toward a wearer of the garment 20. The side-emitting optical fibers 42, 44, 46, 48, 50, 52 can have a core of high-purity polymethyl methacrylate and cladding of a fluorinated polymer. The side-emitting optical fibers 42, 44, 46, 48, 50, 52 can have a 0.5 mm core diameter and an average attenuation (at 650 nm) of 0.4 dB/m. Side-emitting optical fibers having other specifications could also be employed.

Unlike being woven or knitted into the panel similar to a typical yarn that makes up the panel, the side-emitting optical fiber 42-52 is affixed to the inner surface 38 of each panel 22-32 after the panel has been made, e.g., it is an additional step in the manufacturing process. For example, the side-emitting optical fiber 42-52 can be embroidered to the inner surface 38 of each panel 22-32 through the use of embroidery stitches (not shown), which is more particularly described in U.S. patent application Ser. No. 16/877979, filed May 19, 2020. Alternatively, the side-emitting optical fiber 42-52 can be fixed via adhesive to the inner surface 38 of each panel 22-32. By affixing each side-emitting optical fiber 42-52 to the inner surface 38 of each panel 22-32 after the panel has been made, more freedom as to the location and density of the side-emitting optical fiber is available in manufacturing the garment 20.

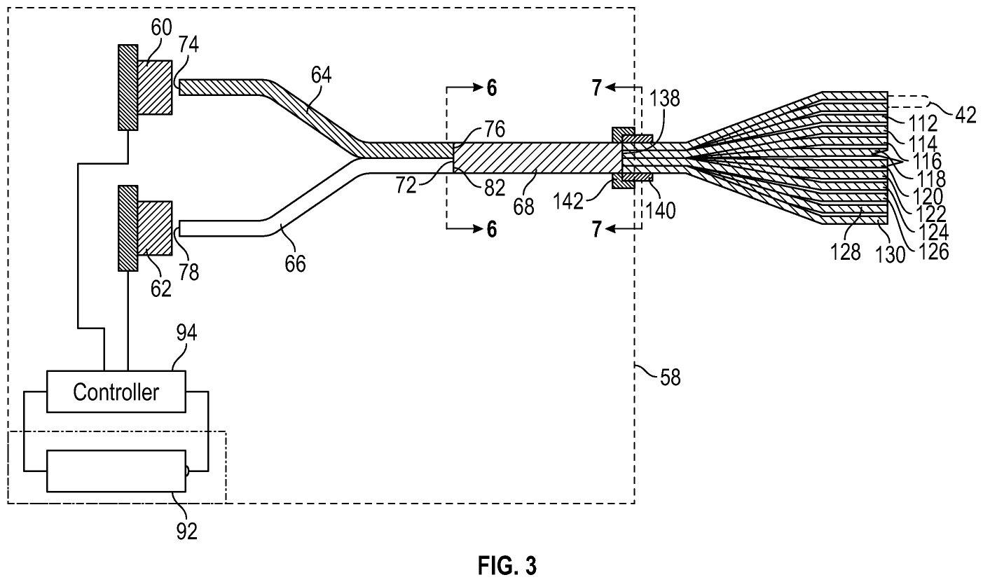

FIG. 2 depicts a light pod 58 attached to the front panel 22 near the right shoulder; however, the light pod 58 could be mounted elsewhere on the garment 20. The garment 20 employs at least one light pipe to distribute light into and, optionally, around the garment 20. FIG. 3 depicts a first optical fiber light source 60, a second optical fiber light source 62, a first wavelength-dedicated light pipe 64, a second wavelength-dedicated light pipe 66, and a combined wavelength light pipe 68 within the light pod 58. The first optical fiber light source 60 emanates light of a first wavelength (e.g., red light between 635 nm-700 nm) and the second optical fiber light source 62 emanates light of a second wavelength (e.g., IR light between 700 nm-1 mm) that is different than the first wavelength. The first optical fiber light source 60 and the second optical fiber light source 62 can emanate light other than red and IR, respectively, however, it can be desirable that they emanate light of different wavelengths. The optical fiber light sources 60, 62 can each be a laser diode, a typical LED, or another type of light source. In the embodiment illustrated in FIG. 3, the optical fiber light sources 60, 62 are each a laser diode.

The first wavelength-dedicated light pipe 64 is positioned between the first optical fiber light source 60 and an upstream end 72 of the combined wavelength light pipe 68. Similarly, the second wavelength-dedicated light pipe 66 is positioned between the second optical fiber light source 62 and the upstream end 72 of the combined wavelength light pipe 68. Both the first wavelength-dedicated light pipe 64 and the second wavelength-dedicated light pipe 66 are made from materials such the each exhibits relatively low light attenuation, which is at least lower than that of each side-emitting optical fiber 42-52. As such, each of the first wavelength-dedicated light pipe 64 and the second wavelength-dedicated light pipe 66 are configured to transmit substantially all of the radiation generated from the respective optical fiber light source 60, 62 to the upstream end 72 of the combined wavelength light pipe 68. Radiation enters the first wavelength-dedicated light pipe 64 at its first end 74 adjacent the first optical fiber light source 60 and travels along the length of the first wavelength-dedicated light pipe 64 via total internal reflection and escapes from its second end 76 into the upstream end 72 of the combined wavelength light pipe 68. Similarly, enters the second wavelength-dedicated light pipe 66 at its first end 78 adjacent the second optical fiber light source 62 and travels along the length of the second wavelength-dedicated light pipe 66 via total internal reflection and escapes from its second end 82 into the upstream end 72 of the combined wavelength light pipe 68. As an example, each of the first wavelength-dedicated light pipe 64 and the second wavelength-dedicated light pipe 66 can be an optical fiber having a core of high-purity polymethyl methacrylate and a cladding of fluorinated polymer. Each of the first wavelength-dedicated light pipe 64 and the second wavelength-dedicated light pipe 66 can be made from a flexible material to allow for routing around the therapeutic garment 20.

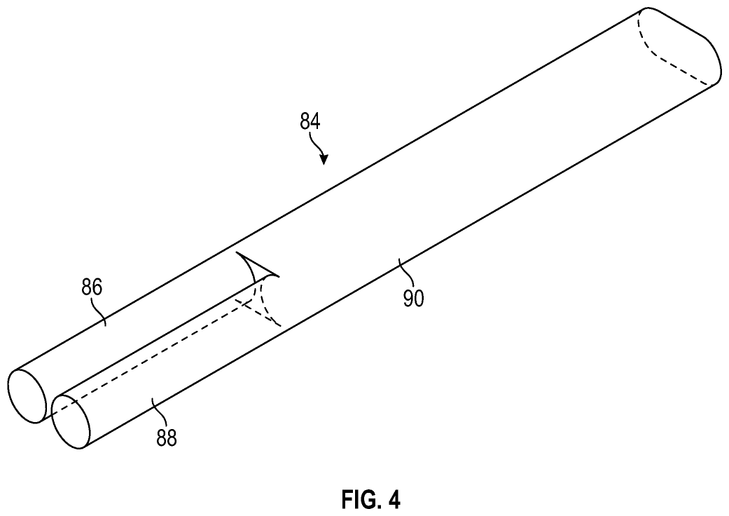

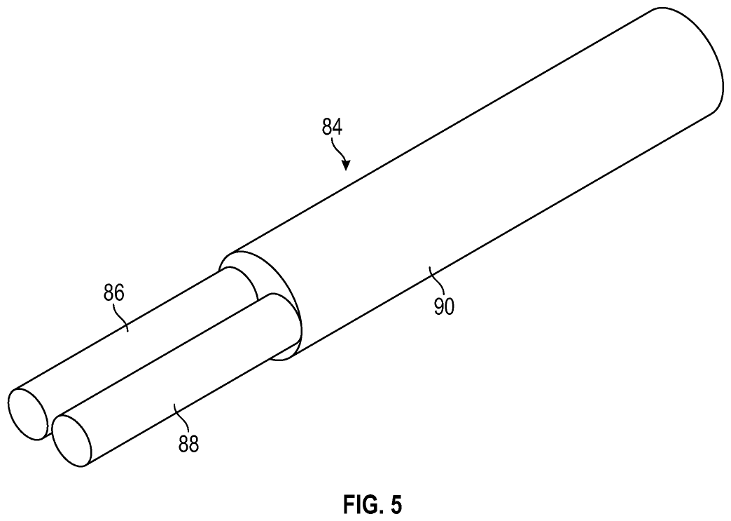

The combined wavelength light pipe 68 can be made from a relatively more rigid material at least as compared to the side-emitting optical fibers 42-52. As an example, the combined wavelength light pipe 68 can be made from a glass or an acrylic material, which can act as a core, and include a reflective jacket. Even though it is referred to as a combined wavelength light pipe 68, it need not combine two wavelengths within it, as will be described in more detail below. With reference to FIGS. 4 and 5, an integrally formed light pipe 84 can be provided with a first wavelength-dedicated light pipe section 86, which can function similarly to the first wavelength-dedicated light pipe 64, a second wavelength-dedicated light pipe section 88, which can function similarly to the second wavelength-dedicated light pipe 66, and a combined wavelength light pipe section 90, which can function similarly to the combined wavelength light pipe 68. As seen when comparing FIGS. 4 and 5, the light pipe sections 86, 88 and 90 can take different cross-sectional configurations, e.g., an elliptical cross section shown in FIG. 4 or a circular cross section show in FIG. 5. Other cross-sectional configurations are contemplated.

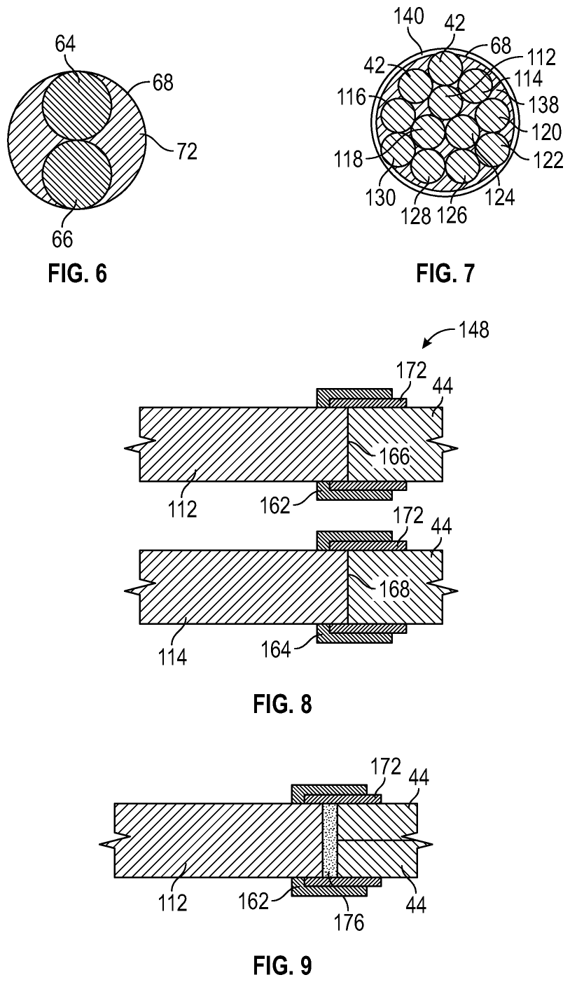

With reference to FIG. 6, each of the first wavelength-dedicated light pipe 64 and the second wavelength-dedicated light pipe 66 is relatively smaller in diameter than combined wavelength light pipe 68. For example, the combined wavelength light pipe 68 has a core having a diameter that is at least two times a respective diameter of each respective core of both the first wavelength-dedicated light pipe 64 and the second wavelength-dedicated light pipe 66.

FIG. 3 further shows a power source 92, which can be a removable rechargeable battery, and a controller 94 in electrical communication with the power source 92, the first optical fiber light source 60 and the second optical fiber light source 62. The controller 94 is configured to control delivery of power to each of the first optical fiber light source 60 and the second optical fiber light source 62. The controller 94 can be configured to control delivery of power to each of the first optical fiber light source 60 and the second optical fiber light source 62 such that only one of the first optical fiber light source 60 and the second optical fiber light source 62 emanates light during a specified period of time. The controller 94 can also be configured to control delivery of power to each of the first optical fiber light source 60 and the second optical fiber light source 62 such that both the first optical fiber light source 60 and the second optical fiber light source 62 emanate light concurrently during a specified period of time.

The light pod 58 depicted in FIG. 3 can combine light from one or more wavelengths into the plurality of side-emitting optical fibers 42-52. This allows for a decreased number of components while allowing for different wavelengths of radiation to be directed toward the wearer of the garment 20. The controller 94 can be configured to pulse light from the first optical fiber light source 60, e.g., red light, for a specified period of time, and then switch off the first optical fiber light source 60 and then switch on the second optical fiber light source 62 to direct IR light toward the wearer of the garment 20. Alternatively, both the first optical fiber light source 60 and the second optical fiber light source 62 can be powered on at the same time. As such, light in different wavelengths can travel through the same plurality of side-emitting optical fibers 42-52 thus reducing the number of fibers necessary to allow for the wearer of the garment to receive the benefits of both wavelengths of light. Additionally, further light sources can be provided and coupled to the combined wavelength light pipe 68, which couples the light sources to the plurality of side-emitting optical fibers 42-52. Also, the optical fiber light sources can be configured to emanate light of wavelengths other than red and IR.

With reference back to FIG. 2, the garment 20 can include light pipe optical fibers 112, 114, 116, 118, 120, 122, 124, 126, 128, 130 optically coupled with a downstream end 138 of the combined wavelength light pipe 68. Each light pipe optical fiber 112-130 can be affixed to the respective fabric panel 22-32 in the same manner that side-emitting optical fibers 42-52 are affixed to the inner surface 38 of each panel 22-32. The light pipe optical fibers 112-130 allow for efficient routing of light throughout the garment 20. The light pipe optical fibers 112-130 can be made from an appropriate material so as to be more flexible than the combined wavelength light pipe 68. For example, the light pipe optical fibers 112-130 can have an allowable bend radius that is at least 36 times the outer diameter of the light pipe optical fiber, which can be a tighter bend radius than that of the combined wavelength light pipe 68. The allowable bend radius is a property of a respective optical fiber. If the respective optical fiber is bent to a radius smaller than the allowable bend radius, then damage can occur to the optical fiber that impacts its ability to transmit light along the length of the optical fiber. Also, each light pipe optical fiber 112-130 can have an allowable bend radius that is about equal, e.g. ±10%, the allowable bend radius of the side-emitting optical fibers 42-52.

FIGS. 3 and 7 depict the side-emitting optical fiber 42 and the light pipe optical fibers 112-130 bundled into a connector 140. With reference to FIG. 3, the connector 140 is received in a socket 142 provided in the light pod 58. Each light pipe optical fiber 112-130 can optically connect with a respective side-emitting optical fiber 44-52. As depicted in FIG. 2, the light pipe optical fiber 112 connects with one end of the side-emitting optical fiber 44 and the light pipe optical fiber 114 connects with the other end of the side-emitting optical fiber 44 through on the back panel 24 via a connection 148. Similarly, the light pipe optical fiber 116 connects with one end of the side-emitting optical fiber 46 and the light pipe optical fiber 118 connects with the other end of the side-emitting optical fiber 46 on the right sleeve panel 26 via a connection 150. With continued reference to FIG. 2, the light pipe optical fiber 120 connects with one end of the side-emitting optical fiber 48 and the light pipe optical fiber 122 connects with the other end of the side-emitting optical fiber 48 on the left sleeve panel 28 via a connection 152. Also shown in FIG. 2, the light pipe optical fiber 124 connects with one end of the side-emitting optical fiber 50 and the light pipe optical fiber 126 connects with the other end of the side-emitting optical fiber 50 on the right hood panel 30 via a connection 154. Also, the light pipe optical fiber 128 connects with one end of the side-emitting optical fiber 52 and the light pipe optical fiber 130 connects with the other end of the side-emitting optical fiber 52 on the left hood panel 32 via a connection 156. In an alternative arrangement, a similar light pipe optical fiber could be provided on the front panel 22 and connect with the side-emitting optical fiber 42 via a connector similar to the connectors 148-156.

In the illustrated embodiment, each end of each of the side-emitting optical fibers 42-52 can receive radiation. FIG. 8 depicts a close up view of the connection 148 shown in FIG. 2. The other connections 150-156 can be similar in configuration. The light pipe optical fibers 112, 114 can each be provided with a respective socket 162, 164 adjacent respective light-exiting ends 166, 168. Each end of the side-emitting optical fiber 42 is provided with a ferrule 172 that is received in the respective sockets 162, 164. In an alternative arrangement, fewer light pipe optical fibers may be provided. For example, only one light pipe optical fiber may be provided for providing radiation to each panel 22-32. FIG. 9 depicts such an example where the light pipe optical fiber 114 is omitted from delivering light to the side-emitting optical fiber 42. The ferrule 172 captures both ends of the side-emitting optical fiber 42. FIG. 9 also depicts an index-matching material 176 that can fill an air gap between the light pipe optical fiber 112 and the side-emitting optical fiber 42. The index-matching material 176 can be a liquid, an adhesive, or a gel having an index of refraction that nearly matches that of the light pipe optical fiber 112 and the side-emitting optical fiber 42. Similar index-matching material can be provided at all other junctions of light generating or light carrying elements.

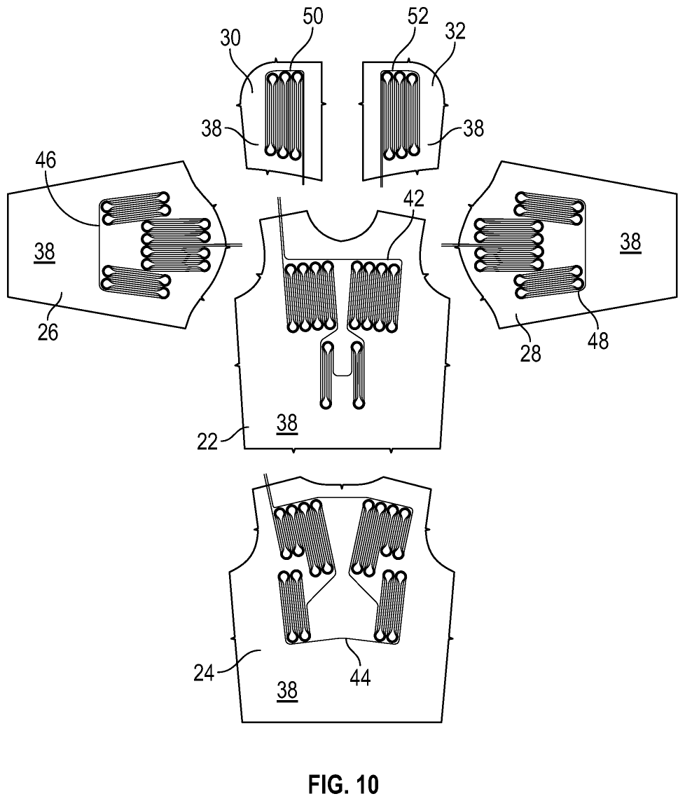

FIG. 10 depicts the panels 22-23 for the garment 20 in FIG. 1 without the light pipe optical fibers 112-130. In this embodiment, only the first optical fiber light source 60 may also be employed. The light pipe 180 can be similar to the combined wavelength light pipe 68 described above. The index-matching material 176 can also be provided between the first optical fiber light source 60 and the upstream end 182 of the light pipe 180 as well as, although not shown, between the downstream end 184 of the light pipe 68 and each end of each of the side-emitting optical fibers 42-52.

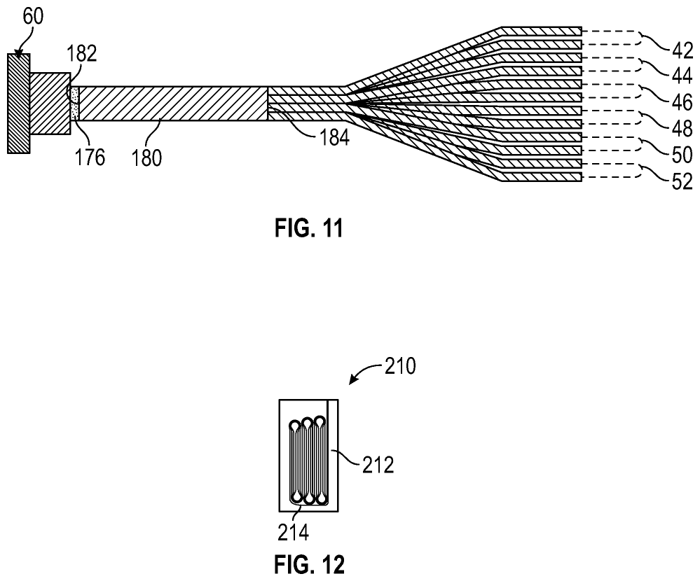

As shown in FIG. 10, the light pipe 180 is relatively larger in diameter than each of the side-emitting optical fibers 42-52. The light pipe 180 optically couples the plurality of side-emitting optical fibers 42-52 to the first optical fiber light source 60. The light pipe 180 includes the upstream end 182 optically coupled to the first optical fiber light source 60 and the downstream end 184 optically coupled to at least one end (and in the illustrated embodiment each end) of each of the plurality of side-emitting optical fibers 42-52. In the illustrated embodiment, the downstream end 184 of the light pipe 180 is butt coupled to each end of each of the plurality of side-emitting optical fibers 42-52. Although not depicted in FIG. 11, the plurality of side-emitting optical fibers 42-52 can be bundled into a ferrule-like connector received in a socket (also not shown) fixed to the light pipe 180 over the downstream end 184 of the light pipe 180.

As mentioned above, the light pipe 180 (as well as the combined wavelength light pipe 68) is relatively larger in diameter than each of the side-emitting optical fibers 42-52. The light pipe 180 can have a core having a diameter that is at least three times a respective diameter of each respective core of each of the plurality of relatively smaller side-emitting optical fibers 42-52. For example and with reference to FIGS. 3 and 11, the light pipe 180 and the combined wavelength light pipe 68 can have a core diameter of greater than 1.5 mm, and each of the plurality of side-emitting optical fibers 42-52 can have a core diameter of 0.5 mm.

Unlike the side-emitting optical fibers 42-52, which are configured to promote leakage of the core transmitted radiation via their side surfaces, the light pipes 64, 66, 68, 180 and the light pipe optical fibers 112-130 are configured to transmit substantially all of the radiation generated from the optical fiber light sources 60, 62 to the side-emitting optical fibers 42-52. Radiation enters the light pipes 64, 66, 68, 180 and the light pipe optical fibers 112-130 at a proximal end, with respect to the optical fiber light sources 60, 62, and travels along the length of each via total internal reflection and escapes from a distal end into each of the side-emitting optical fibers 42-52. Bundling the plurality of side-emitting optical fibers 42-52 or the light pipe optical fibers 112-130 and butt coupling them to a relatively larger light pipe 68 or 180 improves the consistency of light entering each end of the respective ends of the side-emitting optical fibers 42-52 as compared to each end of the side-emitting optical fibers 42-52 being directly coupled to the optical fiber light source 60 or 62. Light entering the relatively larger light pipe 68 or 180 is allowed to enter the light pipe's different modes and consistently couple to the relatively smaller side-emitting optical fibers 42-52 or the light pipe optical fibers 112-130. The length of the side-emitting optical fibers 42-52 or the light pipe optical fibers 112-130 can be changed to allow for routing through the garment 20 and may include routing of fabric panels and other coverage areas. Providing the relatively larger light pipe 68 or 180 may also decrease manufacturing and/or assembly precision, result in potential efficiency gains, and less reliance on balancing light in each fiber arrangement.

As mentioned above, the light therapy wearable can take different forms. As another example, FIG. 12 depicts a wearable 210 for providing light therapy including a fabric panel 212 and a side-emitting optical fiber 214 affixed to the fabric panel 212. The wearable 210 can be in the form of a wrap intended to be wrapped around a body part. The side-emitting optical fiber 214 can receive light from an optical fiber light source (not shown in FIG. 12, but similar to either optical fiber light sources 60, 62) via a light pipe including a proximal end, with respect to the optical fiber light source, optically coupled to the optical fiber light source and a distal end optically coupled to a light-receiving end of the side-emitting optical fiber 214. The light pipe can be similar to any of the light pipes 64, 66, 68, 180 or the light pipe optical fibers 112-130 described above.

Therapeutic wearables have been described above in particularity. Modifications and alterations will occur to those upon reading and understanding the preceding detailed description. The invention, however, is not limited to only the embodiments described above. Instead, the invention is broadly defined by the appended claims and the equivalents thereof. It will be appreciated that various features of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A wearable for providing light therapy to a wearer, the wearable comprising:
a fabric panel;
a first optical fiber light source emanating light of a first wavelength;
a second optical fiber light source emanating light of a second wavelength that is different than the first wavelength;
a side-emitting optical fiber affixed to the fabric panel;
a combined wavelength light pipe including a downstream end optically coupled with the side-emitting optical fiber;
a first wavelength-dedicated light pipe including a proximal end, with respect to the first optical fiber light source, optically coupled to the first optical fiber light source and a distal end optically coupled to the combined wavelength light pipe, the first wavelength-dedicated light pipe being positioned between the first optical fiber light source and an upstream end of the combined wavelength light pipe; and
a second wavelength-dedicated light pipe including a proximal end, with respect to the second optical fiber light source, optically coupled to the second optical fiber light source and a distal end optically coupled to the combined wavelength light pipe, the second wavelength-dedicated light pipe being positioned between the second optical fiber light source and the upstream end of the combined wavelength light pipe.

2. (canceled)

3. The wearable of claim 1, wherein the combined wavelength light pipe has a core having a diameter that is at least two times a respective diameter of each respective core of both the first wavelength-dedicated light pipe and the second wavelength-dedicated light pipe.

4. The wearable of claim 1, further comprising at least one light pipe optical fiber optically coupled with the downstream end of the combined wavelength light pipe, the at least one light pipe optical fiber having lower light attenuation along its length as compared to the side-emitting optical fiber.

5. The wearable of claim 4, wherein the at least one light pipe optical fiber is affixed to the fabric panel.

6. The wearable of claim 4, wherein the at least one light pipe optical fiber has a smaller bending radius than the combined wavelength light pipe.

7. The wearable of claim 4, wherein the combined wavelength light pipe includes a core having a diameter that is at least three times a diameter of a core of the at least one light pipe optical fiber.

8. The wearable of claim 4, wherein the at least one light pipe optical fiber and the side-emitting optical fiber are about equal in outer diameter.

9. The wearable of claim 4, wherein the at least one side-emitting optical fiber includes a plurality of side-emitting optical fibers, and
the at least one light pipe optical fiber includes a light-entering end optically coupled with the downstream end of the combined wavelength light pipe and a light-exiting end optically coupled with a respective light-entering end of at least one side-emitting optical fiber among the plurality of side-emitting optical fibers.

10. The wearable of claim 9, wherein the at least one light pipe optical fiber includes a plurality of light pipe optical fibers, and respective light-entering ends of each light pipe optical fiber being optically coupled with the combined wavelength light pipe at the downstream end.

11. The wearable of claim 10, wherein respective light-exiting ends of each light pipe optical fiber being optically coupled with respective light-receiving ends of each side-emitting optical fiber among the plurality of side-emitting optical fibers.

12. The wearable of claim 11, wherein each end of at least one of the plurality of side-emitting optical fibers is butt coupled to the combined wavelength light pipe at the downstream end.

13. The wearable of claim 1, wherein the distal end of the combined wavelength light pipe is optically coupled to each end of the side-emitting optical fiber.

14. The wearable of claim 1, wherein the side-emitting optical fiber is one of a plurality of side-emitting optical fibers, and
the distal end of the combined wavelength light pipe is optically coupled to each end of each of the plurality of side-emitting optical fibers.

15. The wearable of claim 14, wherein the distal end of the combined wavelength light pipe is butt coupled to each end of each of the plurality of side-emitting optical fibers.

16. (canceled)

17. The wearable of claim 22, wherein the light pipe is one of an upstream light pipe and at least one downstream light pipe optically coupled to the upstream light pipe.

18. The wearable of claim 17, wherein the upstream light pipe includes an upstream end or a first end that coincides with the proximal end of the light pipe and a downstream end or a second end, and each downstream light pipe of the at least one downstream light pipe includes a respective light-entering end optically coupled to the downstream end or to the second end of the upstream light pipe.

19. The wearable of claim 17, wherein the side-emitting optical fiber is one of a plurality of side-emitting optical fibers, and
wherein each downstream light pipe includes a light-exiting end optically coupled to a respective light-entering end of each side-emitting optical fiber of the at least one side-emitting optical fiber.

20. The wearable of claim 22, wherein the light pipe is one of a plurality of light pipe optical fibers each affixed to the fabric panel.

21. The wearable of claim 1, wherein the light pipe and the side-emitting optical fiber are about equal in outer diameter.

22. A wearable for providing light therapy to a wearer, the wearable comprising:
a fabric panel;
an optical fiber light source;
a plurality of side-emitting optical fibers affixed to the fabric panel; and
a light pipe including a proximal end, with respect to the optical fiber light source, optically coupled to the optical fiber light source and a distal end optically coupled to a respective light-receiving end of each side-emitting optical fiber, wherein the light pipe has a core having a diameter that is at least three times a respective diameter of each respective core of each of the plurality of side-emitting optical fibers.
